(19) United States
(12) Patent Application Publication
(10) Pub. No.: US 2023/0399602 A1
(43) Pub. Date: Dec. 14, 2023

(54) Self-Contained Biological Indicator With Integrated Activation Control (AC-SCBI)

(71) Applicant: GKE-GMBH, Waldems (DE)

(72) Inventor: Ulrich Kaiser, Waldems (DE)

(21) Appl. No.: 18/036,327

(22) PCT Filed: Jan. 8, 2021
(86) PCT No.: PCT/EP2021/050293

§ 371 Date: May 10, 2023

(51) Int. Cl. C12M 1/12; C12Q 1/22; G01N 21/78

(52) U.S. Cl. CPC C12M 37/06; C12Q 1/22; G01N 21/78

(57) ABSTRACT

The invention relates to a self-contained biological indicator comprising a vial, an ampule arranged inside said vial, a growth medium for growing microorganisms, the growth medium being arranged inside the ampule and the growth medium having a first color, a biological indicator, a carrier for holding the biological indicator, wherein the biological indicator is arranged on the carrier, and at least one color change indicator having a second color, wherein the at least one color change indicator is arranged inside the vial but outside the ampoule, the growth medium does not contain any of the at least one color change indicator and the first color of the growth medium is changed by the second color of the at least one color change indicator when mixed with the at least one color change indicator.

The invention relates to a modified design of a self-contained biological indicator (SCBI).

### BACKGROUND OF THE INVENTION

Standard Biological indicators (BIs) are made using alive spores of germs, which are more resistant to sterilization processes than pathogenic germs. The spores of the germs are inoculated on carrier discs or strips made from paper, stainless steel, glass fiber or other non-woven materials and packed in sterile proof pouches.

BIs are used to monitor the success of a sterilization process. They are applied together with the goods inside of a sterilization chamber. At the end of the sterilization process the BIs are taken out and have to be checked if the spores of the BIs are killed or are still alive, which is not visible directly on the BI. Historically they are sent into a microbiological laboratory and transferred aseptically into a growth medium which is incubated afterwards. After some time, it can be observed if the spores are killed (no growth) or are still alive (growth). If they grow, the sterilization process was not successful and a report is sent from the laboratory to the user making sterile products.

To prevent bringing the sterilized BIs into a microbiological laboratory, so-called self-contained biological indicators (SCBIs) have been developed and are sold from various companies in the market. Examples for such SCBIs can be found in U.S. Pat. Nos. 5,552,320 and 3,440,144 A. The SCBI according to U.S. Pat. No. 5,552,320 A may contain a color change indicator inside an ampoule further containing medium for neutralizing hydrogen peroxide. U.S. Pat. No. 5,073,488 A discloses a method for determining the efficacy of a sterilization cycle using an enzyme source. Such enzymes are also disclosed in WO 2010/079357 A1.

SCBIs normally have a plastic vial containing the BI and a glass ampoule with a growth medium inside, wherein the growth medium contains a color-indicator. Before incubation inside a heated compartment (which is also referred to as an incubator) at temperatures between 35° C. and 60° C., the SCBIs require to be activated. The activation is carried out cracking the glass ampoule inside the vial of the SCBI by squeezing the elastic plastic vial. The BI gets now in contact with the growth medium and is able to grow. If spores are alive, they will grow after some time inside the liquid growth medium and produce color change ingredients in the liquid.

The produced metabolites are changing color to signal growth of the BI and therefore a non-successful sterilization process is detected.

The advantage of SCBIs is that no microbiological laboratory is required to provide the result of the sterilization process. The user can incubate the SCBI themselves getting much quicker information about the result of the sterilization process. This procedure is the current state of the art worldwide.

It can also be provided that the SCBI is usable for testing that the sterility is achieved by radiation, such as UV radiation and gamma radiation, but also by gas sterilization, such as ethylene oxide sterilization, formaldehyde sterilization (LTSF) and steam.

Hereby, the SCBI is usable for a great variety of sterilization techniques.

A standard SCBI is schematically shown in FIG. 1. The SCBI 1 according to FIG. 1 contains a mixture 2 of a growth medium and a color change indicator (CCI). The SCBI 1 further contains a BI 3 which is applied on a carrier. The mixture 2 of the growth medium and the CCI is arranged inside a glass ampoule 4. The glass ampoule 4 and the carrier are arranged inside a plastic vial 5 of the SCBI 1. The plastic vial 5 is closed to the outside by a cap 7. The cap 7 has a hole for the passage to allow that the sterilization gas can penetrate into the SCBI to kill the BI 3. The sterilization gas penetration is enforced by vacuum pressure pulses of the sterilization process. In addition, a filter 6 for preventing external microorganisms to enter into the plastic vial 5 is installed.

The SCBI 1 is sterilized together with other equipment and is taken out of the sterilizer. Then this SCBI is activated by breaking the glass ampoule inside the SCBI. Then the SCBI is incubated in an incubator at 35-60° C. depending on the spore type used, whereby the mixture 2 gets in contact with the BI 3. If microorganisms are alive in the BI 3 they will grow in the growth medium producing metabolites and thereby changing the color of the CCI, which is visible from the color change of the liquid 2.

### Problem Description

SCBIs are sold worldwide on the market. It is common practice to perform also a positive control with an unsterilized SCBI in order to check its viability to grow: An unsterilized SCBI shall grow and should show a color change after incubation. Quite often it is reported that these SCBIs, where the positive control is carried out, do not change color even though they have not been sterilized and should show a color change because they contain alive spores. The actual problem is that users have not activated the SCBI in advance by crushing the glass ampoule with the growth medium. Therefore, the BI does not get in contact with the growth medium and cannot grow during incubation providing a misleading pass result because no color change signals normally a pass result of the sterilization process. If the problem is not detected that no activation of the SCBI has been carried out, non-sterile goods could be released at the end of a sterilization process, which could cause non-sterile operations leading to infections in hospitals.

The object of the present invention is to overcome these and other disadvantages of presently known SCBIs and methods for using SCBIs.

The object of the present invention is thus to provide an SCBI and a method for using an SCBI by which a sterilization can be securely detected. The danger of wrong SCBI handling will be eliminated so that no activation is carried out. The cost of the SCBI or the method should be as low as possible. The results should be easy to interpret and the SCBI and the method should be usable easily with low effort.

### SUMMARY OF THE INVENTION

The invention solves the problem by an SCBI according to claim 1 and by a method according to claim 16. Preferred embodiments are described in claims 2 to 15, depending on claim 1, and in claim 17, depending on claim 16.

In more detail the invention solves problems by an SCBI comprising a vial, an ampoule arranged inside said vial, a growth medium for growing microorganisms, said growth medium being arranged inside said ampoule and said growth medium having a first color, a biological indicator, a carrier for holding said biological indicator, wherein said biological indicator is arranged on said carrier, and at least one color change indicator having a second color, wherein the at least one color change indicator is arranged inside said vial but outside said ampoule, the growth medium inside the ampoule does not contain any of the at least one color change indicator and the first color of the growth medium is changed by the second color of the at least one color change indicator when the growth medium is mixed with the at least one color change indicator (CCI).

Preferably, the ampoule is made of a frangible material such as glass.

It can be provided that the biological indicator comprises living microorganisms, preferably spores of germs.

The first color preferably is a transparent liquid and/or a pastel color and may be of a whitish appearance, while the second color and the indication color are preferably of a stronger contrast. Therefore, according to the present invention the first color can be transparent. The indication color can be visible with the naked eye or in combination with UV-light by means of luminescence.

The present invention is not to put the at least one CCI inside the growth medium but to put it inside the vial, outside the ampoule, outside of the growth medium. This can be carried out in different ways, as described and claimed in the sub-claims 2 to 5.

An alternative SCBI design has been patented in U.S. Pat. No. 5,073,488 or in the patent application WO 2010/079357 A1 where the growth medium contains instead of a pH-indicator a dissolved chemical substance which interacts after sterilization and activation with enzymes on the surface of the BI. When BIs remain alive after sterilization, the enzymes react with the chemical to provide a bioluminescence reaction which can be detected using ultraviolet light (UV-light). When using this detection method, the chemical substance as claimed in claim 6 also has to be separated from the growth medium.

The growth medium is preferably a liquid or viscous growth medium.

Preferably, the vial is deformable or a plug is pushable inside the vial for opening the ampoule with growth medium.

It can be provided, that the at least one CCI is arranged on the carrier together with the biological indicator.

Hereby it is achieved that it can be ensured that the change of the first color of the growth medium by mixing with the CCI independent at which of the 4 different areas it is positioned will automatically take place, when mixed with the growth medium.

The color indicator may be integrated as part of the BI carrier inside the vial but outside the growth medium. However, the CCI could hinder quick growth, if in close contact with the BI on the carrier.

Alternatively, it can be provided that the CCI is separately arranged inside the vial 5 as one pearl or on one separate carrier so there is no contact to the biological indicator thereby preventing a negative influence of the growth of microorganisms or a negative influence on the long-term stability of the biological indicator on the carrier.

This embodiment has the advantage that the CCI does not hinder quick growth of a still alive BI when coming out of the sterilization process, but it could still be possible to mix the growth medium with the CCI.

Hereby it can be provided that the carrier of the biological indicator and the pearl of the CCI or the separate carrier carrying the CCI are separated from each other inside the vial, such that no interaction between biological indicator and the at least one CCI occurs during sterilization.

Further it can be provided that the CCI is arranged on an inner surface of a wall inside the vial 5 or on an outer surface of a wall of the glass ampoule 4, preferably without being in contact with the biological indicator on the carrier.

During production of the SCBI the CCI is inserted into the deformable vial before the SCBI is finally assembled. The CCI is dissolved in a liquid with high vapor pressure and injected into the vial before the BI plate is inserted. The liquid evaporates quickly and the CCI adheres on the plastic vial walls. Alternatively, the glass ampoule can be sprayed or immersed in the dissolved solution of the CCI and afterwards dried before the glass ampoule is inside the SCBI. Then the CCI adheres on the outside walls of the glass ampoule which is then inserted into the SCBI plastic vial and the SCBI is then finally assembled. Alternatively or additionally the CCI can be arranged inside a second ampoule, which is arranged inside the vial in addition.

It can also be provided that the CCI changes its color due to an enzymatic reaction together with a chemical substance, wherein preferably the color change, an optical density change or a biofluorescence reaction with UV-light of the CCI is detectable.

It can also be provided that the carrier is a carrier disc or a strip and/or the carrier is made from paper, stainless steel or glass fiber or any plastic material.

These embodiments for the carrier are easy to handle and are functionally sufficient.

It can also be provided that the at least one CCI is a pH-indicator.

These CCIs are reliable, cost efficient and show a strong change of colors.

It can also be provided that the first color of the growth medium is colorless, clear or transparent.

Hereby, the first color can be easily and clearly changed by the second color of the CCI. This reduces the danger of wrong visible identification of the state (activated or not activated) of the SCBI after incubation.

The objects of the present invention are also met by a method for testing the sterility using an SCBI, comprising the chronological steps which are explained in the separate flow chart shown in FIG. 8.

The objects of the present invention are also met by a method for testing the sterility using an SCBI, comprising the chronological steps:
A) Activating an SCBI transferring a growth medium inside a vial;
B) Bringing the growth medium in contact with a biological indicator and with CCI, wherein the color of the growth medium is changed by the CCI;
C) Allowing microorganisms from the biological indicator to grow in the growth medium; and
D) Observing by visual or photometric inspection, if a change of color occurs which could be a result of a reaction of the at least one CCI with at least one metabolic product from the microorganism.

Preferably, the method step A) is achieved by deforming the vial to break the ampoule.

It can be provided that the method is performed using an SCBI according to the present invention as discussed above.

Hereby, the method has the benefits of the SCBI according to the present invention.

It can also be provided that before method step C) is carried out, controlling if a change of the color of the growth medium occurred and only if the change of color is observed continuing with step C) and otherwise further opening the ampoule or deforming the vial to allow mixing of the growth medium with the CCI.

Hereby, it is achieved that the danger of possible false results is eliminated.

The invention is based on the surprising finding that a breaking of the ampoule containing the growth medium can be controlled by a color change of the growth medium, which results from a mixture with the at least one CCI, which at least one CCI is used later for controlling the growth of microorganisms. Hereby, the user has a visible control if the SCBI is been prepared for incubation (activated) putting growth medium and CCI together. As the user is already prepared to check the color to interpret the result of the test, this is the most easy and efficient way to control the function of the SCBI.

To prevent the possible user mistake of not bringing the SCBI in the correct working condition, the invention separates the pH-indicator from the growth medium but leaves it inside the SCBI vial. The growth medium will preferably remain clear transparent, colorless or pastel colored, without any color or only weak and clearly distinguishable color inside the growth medium. If the incubation is carried out without activation, which means without having broken open the glass ampoule, the growth medium will remain the first color (preferably colorless) indicating that the activation step had not been carried out properly before incubation. After the SCBI is activated by breaking open the glass ampoule, the color indicator outside the growth medium but inside the vial of the SCBI dissolves in the growth medium, thereby changing the color of the growth medium to the second color, for example a purple indicating activation. During incubation when no further color change occurs, the sterilization process was successful. A color change to the indication color, for example to yellow, signals an unsuccessful sterilization process. (see enclosed flow chart— FIG. 8)

The user can visually see and check if an SCBI has been activated before incubation or not. If the growth medium remains in the first color (clear transparent), the SCBI has not been activated.

It is a big risk if users do not activate an SCBI, but incubate after sterilization without activation and therefore cannot detect a color change. No color change signals a successful sterilization process. However, the BI inside the SCBI cannot grow, even remaining alive, since the growth medium is missing. If this mistake is not detected before sterilization release, false positive results may occur with a high risk that a non-sterile load is released with the consequence non-sterile operations may be carried out in a hospital causing infection of a patient.

This mistake is easily detected when the user after incubation detects having a clear growth medium or a growth medium in the first color inside the SCBI vial and no result can be achieved because of the missing mixture of growth medium with the BI.

Using the modified SCBI according to the invention after the SCBI is activated, the CCI color becomes visible in the growth medium indicating that a correct activation has been carried out before incubation and the later result will be valid, since no wrong result due to not activated SCBI can occur.

It is preferred that not only the first color clearly deviates from the second color but also from the indication color to allow recognizing if the SCBI has been activated after incubation of the SCBI.

Incubation after sterilization takes up to 7 days and is therefore time-critical, because before the sterilization result is not available the sterilized load cannot be released. In case no activation has been carried out but the mistake has been observed before load release, the first incubation time is wasted requiring to start a second incubation process with increased time required until sterile product release can occur.

This invention reduces the risk of incorrectly executed incubation procedures with SCBIs remaining undetected dramatically. The use of SCBIs for testing sterilization processes becomes much safer and much more reliable.

### BRIEF DISCUSSION OF THE FIGURES

FIG. 1 shows a standard SCBI according to the state of the art with a pH-indicator or other CCI inside a glass ampoule together with a growth medium;

FIG. 2 shows an SCBI according to a first embodiment according to the present invention having a CCI outside a glass ampoule on a carrier with BI;

FIG. 3 shows an SCBI according to a second embodiment according to the present invention having a color change indicator arranged as a pearl at the bottom area of the plastic vial;

FIG. 4 shows an SCBI according to a third embodiment according to the present invention having a CCI arranged at the surface inside the SCBI plastic vial;

FIG. 5 shows an SCBI according to a fourth embodiment according to the present invention having a CCI arranged at the outside glass ampoule surface;

FIG. 6 shows an SCBI according to a fifth embodiment according to the present invention having a CCI arranged in a second glass ampoule below or above the glass ampoule containing the growth medium;

FIG. 7 shows an SCBI according to a sixth embodiment according to the present invention having a CCI arranged on a piece of paper inside the plastic vial, outside of the glass ampoule.

FIG. 8 shows an exemplary sequence diagram for a method according to the present invention.

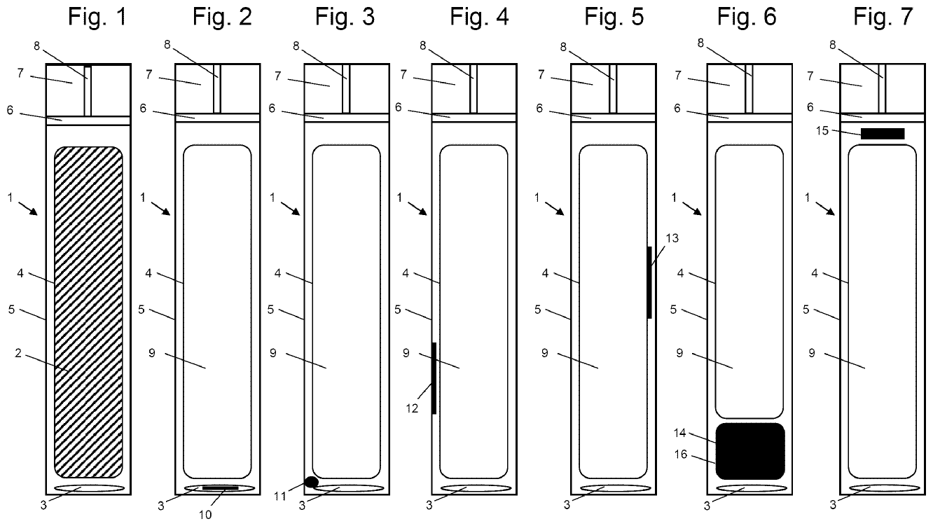

### DETAILED DESCRIPTION OF THE INVENTION

FIG. 2 shows an SCBI 1 according to the present invention. The SCSI 1 comprises a growth medium 9 which is contained inside an ampoule 4. The growth medium 9 can be colorless, transparent or of a white appearance. The ampoule 4 can be translucent. The ampoule 4 is contained in a vial 5. The ampoule 4 can be made of glass or another manually breakable material. The vial 5 can be made of a plastic which is deformable, preferably from an elastically deformable plastic. The vial 5 can be deformable such as to allow breaking of the ampoule 4 inside the vial 5. The vial 5 is translucent to allow visual control of the color of its ingot.

A biological indicator 3 (BI) can be arranged on a carrier inside the vial 5 but outside the ampoule 4 so that no physical contact between the BI 3 and the growth medium 9 can occur. The vial 5 can be closed to the outside by means of a cap 7. The cap 7 can comprise filter 6 being permeable for gases but being impermeable for microorganisms and a passage 8 for allowing a gas exchange through cap 7.

According to the present invention, at least one CCI 10 is arranged inside the vial 5 but outside the ampoule 4. The CCI 10 is arranged on the carrier. Alternatively, the CCI could also be located on a separate carrier apart from the carrier for the BI 3.

FIG. 3 shows a second embodiment of an SCBI 1 according to the present invention. The SCBI 1 comprises a growth medium 9 which is contained inside an ampoule 4. The growth medium 9 can be colorless, transparent or of a white appearance. The ampoule 4 can be translucent. The ampoule 4 is contained in a vial 5. The ampoule 4 can be made of glass or another manually breakable material. The vial 5 can be made of a plastic which is deformable, preferably from an elastically deformable plastic. The vial 5 can be deformable such as to allow breaking of the ampoule 4 inside the vial 5. The vial 5 is translucent to allow visual control of the color of its ingot.

A biological indicator 3 (BI) can be arranged on a carrier inside the vial 5 but outside the ampoule 4 so that no physical contact between the BI 3 and the growth medium 9 can occur. The vial 5 can be closed to the outside by means of a cap 7. The cap 7 can comprise filter 6 being permeable for gases but being impermeable for microorganisms and a passage 8 for allowing a gas exchange through cap 7.

According to the present invention, at least one CCI 11 is arranged inside the vial 5 but outside the ampoule 4. The CCI 11 is formed as at least one pearl inside the vial 5 but outside the ampoule 4. Alternatively, the CCI could also be located on a separate carrier apart from the carrier for the BI 3.

FIG. 4 shows a third embodiment of an SCBI 1 according to the present invention. The SCBI 1 comprises a growth medium 9 which is contained inside an ampoule 4. The growth medium 9 can be colorless, transparent or of a white appearance. The ampoule 4 can be translucent. The ampoule 4 is contained in a vial 5. The ampoule 4 can be made of glass or another manually breakable material. The vial 5 can be made of a plastic which is deformable, preferably from an elastically deformable plastic. The vial 5 can be deformable such as to allow breaking of the ampoule 4 inside the vial 5. The vial 5 is translucent to allow visual control of the color of its ingot.

A biological indicator 3 (BI) can be arranged on a carrier inside the vial 5 but outside the ampoule 4 so that no physical contact between the BI 3 and the growth medium 9 can occur. The vial 5 can be closed to the outside by means of a cap 7. The cap 7 can comprise filter 6 being permeable for gases but being impermeable for microorganisms and a passage 8 for allowing a gas exchange through cap 7.

According to the present invention, at least one CCI 12 is arranged inside the vial 5 but outside the ampoule 4. The CCI 12 is arranged on the inner side of a wall of the vial 5. Alternatively, the CCI could also be located on a separate carrier apart from the carrier for the BI 3.

FIG. 5 shows a fourth embodiment of an SCBI 1 according to the present invention. The SCBI 1 comprises a growth medium 9 which is contained inside an ampoule 4. The growth medium 9 can be colorless, transparent or of a white appearance. The ampoule 4 can be translucent. The ampoule 4 is contained in a vial 5. The ampoule 4 can be made of glass or another manually breakable material. The vial 5 can be made of a plastic which is deformable, preferably from an elastically deformable plastic. The vial 5 can be deformable such as to allow breaking of the ampoule 4 inside the vial 5. The vial 5 is translucent to allow visual control of the color of its ingot.

A biological indicator 3 (BI) can be arranged on a carrier inside the vial 5 but outside the ampoule 4 so that no physical contact between the BI 93 and the growth medium 9 can occur. The vial 5 can be closed to the outside by means of a cap 7. The cap 7 can comprise filter 6 being permeable for gases but being impermeable for microorganisms and a passage 8 for allowing a gas exchange through cap 7.

According to the present invention, at least one CCI 13 is arranged inside the vial 5 but outside the ampoule 4. The CCI 13 is arranged on the outer side of a wall of the ampoule 4. Alternatively, the CCI could also be located on a separate carrier apart from the carrier for the BI 3.

FIG. 6 shows a fifth embodiment of an SCBI 1 according to the present invention. The SCBI 1 comprises a growth medium 9 which is contained inside an ampoule 4. The growth medium 9 can be colorless, transparent or of a white appearance. The ampoule 4 can be translucent. The ampoule 4 is contained in a vial 5. The ampoule 4 can be made of glass or another manually breakable material. The vial 5 can be made of a plastic which is deformable, preferably from an elastically deformable plastic. The vial 5 can be deformable such as to allow breaking of the ampoule 4 inside the vial 5. The vial 5 is translucent to allow visual control of the color of its ingot.

A biological indicator 3 (BI) can be arranged on a carrier inside the vial 5 but outside the ampoule 4 so that no physical contact between the BI 3 and the growth medium 9 can occur. The vial 5 can be closed to the outside by means of a cap 7. The cap 7 can comprise filter 6 being permeable for gases but being impermeable for microorganisms and a passage 8 for allowing a gas exchange through cap 7.

According to the present invention, at least one CCI 14 is arranged inside the vial 5 but outside the ampoule 4. The CCI 14 is arranged in a second ampoule 16, which is arranged inside the vial 5. Alternatively, the CCI could also be located on a separate carrier apart from the carrier for the BI 3.

FIG. 7 shows a sixth embodiment of an SCBI 1 according to the present invention. The SCBI 1 comprises a growth medium 9 which is contained inside an ampoule 4. The growth medium 9 can be colorless, transparent or of a white appearance. The ampoule 4 can be translucent. The ampoule 4 is contained in a vial 5. The ampoule 4 can be made of glass or another manually breakable material. The vial 5 can be made of a plastic which is deformable, preferably from an elastically deformable plastic. The vial 5 can be deformable such as to allow breaking of the ampoule 4 inside the vial 5. The vial 5 is translucent to allow visual control of the color of its ingot.

A biological indicator 3 (BI) can be arranged on a carrier inside the vial 5 but outside the ampoule 4 so that no physical contact between the BI 3 and the growth medium 9 can occur. The vial 5 can be closed to the outside by means of a cap 7. The cap 7 can comprise filter 6 being permeable for gases but being impermeable for microorganisms and a passage 8 for allowing a gas exchange through cap 7.

According to the present invention, at least one CCI 15 is arranged inside the vial 5 but outside the ampoule 4. The CCI 15 is arranged on a paper, which is arranged inside the vial 5. Alternatively, the CCI could also be located on a separate carrier apart from the carrier for the BI 3.

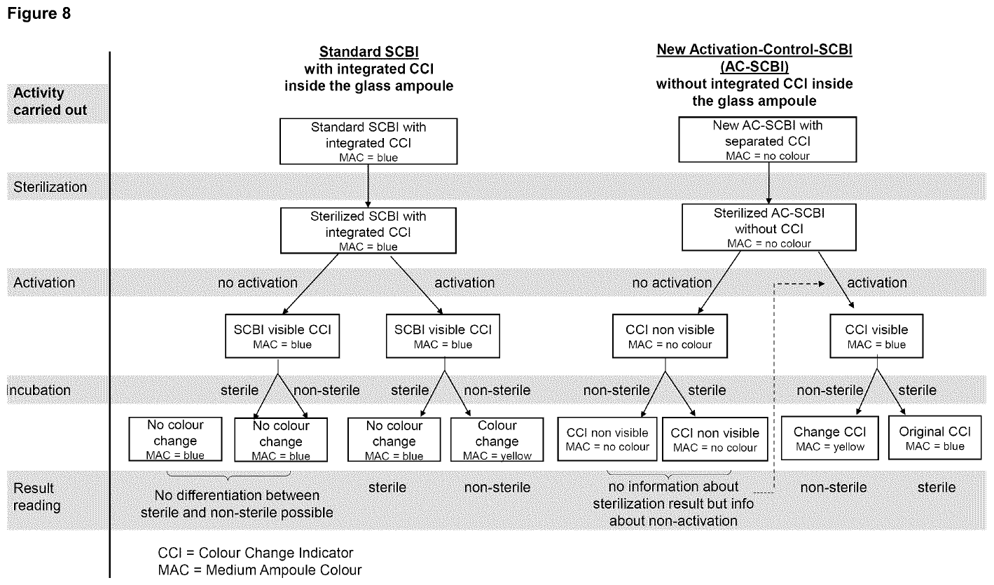

FIG. 8 shows a method according to the present invention for using an SCBI as an exemplary sequence diagram on the right hand side. On the left hand side of FIG. 8 a method according to the state of the art is shown as a comparison. The method is discussed using an SCBI according to FIG. 2. After having used the SCBI 1 for a sterilizing cycle of any kind (gas sterilization or radiation sterilization), the ampoule 4 is broken inside the vial 5 of the SCBI 1 by deforming the vial 5. The liquid or pasty growth medium 9 flows from the broken ampoule 4 and mixes with the CCI 10 on the carrier and with the BI 3. Thereby, the at least one CCI 10 changes the color of the growth medium 9. By checking the color change of the growth medium 9 it can be checked if the growth medium 9 has been mixed with the at least one CCI 10 and with the BI 3. If not, the ampoule 4 has not been broken open and this can be taken care of.

If yes, the microorganisms of the BI 3 can grow in the growth medium 9 by processing it for a certain time at an adequate temperature suitable for growth of the microorganisms of the BI 3. Due to metabolic processes of the microorganisms, acid and/or other metabolic products are produced by the growing microorganisms. This acid or the other metabolic products result in a chemical reaction with the at least one CCI 10 which leads to a change in color. Thus, it can be checked if living BIs have been present by checking the color of the ingot of the so processed SCBI 1.

To prevent the possible user mistake of not bringing the SCBI 1 in the correct working condition, the invention separates the CCI 10-15 (like a pH-indicator) from the growth medium 9 but leaves it inside the vial 5. The growth medium 9 will preferably remain clear transparent without any color inside the growth medium 9 in this case. If the incubation is carried out without activation, that means without having destroyed the ampoule 4, the growth medium 9 will remain colorless indicating that the activation step had not been carried out before incubation. After the SCBI 1 is activated by cracking the ampoule 4, the CCI 10-15 (pH-indicator) outside the growth medium 9 but inside the vial 5 is getting dissolved in the liquid growth medium 9 changing the color of the solution, for example purple indicating activation. During incubation when no further color change occurs, the sterilization process was successful, opposite a color change, for example to yellow, signals an unsuccessful sterilization process.

The features of the invention disclosed in the above description, the claims, figures, and exemplary embodiments can be essential both individually and in any combination for implementing the various embodiments of the invention.

### LIST OF REFERENCE NUMBERS

1 Self-contained biological indicator (SCBI)
2 Mixture of growth medium with pH-indicator
3 Biological indicator (BI)
4 Ampoule
5 Vial
6 Filter
7 Cap
8 Passage
9 Growth medium
10 Color change indicator (CCI) on the carrier
11 Pearl of color change indicator
12 Color change indicator on the vial
13 Color change indicator on the ampoule
14 Color change indicator in separate ampoule
15 Color change indicator on paper
16 Second ampoule

1. A self-contained biological indicator (SCBI) comprising
a vial,
an ampule arranged inside said vial, the ampule containing a growth medium for growing microorganisms,
a liquid of the growth medium containing a first color or is transparent,
a biological indicator (BI) on a carrier, and
at least one color change indicator (CCI) having a second color, wherein:
the at least one CCI is arranged inside said vial but outside said ampule, the growth medium inside the ampule does not contain any of the at least one CCI and the first color of the growth medium is changed by the second color of the at least one CCI when the growth medium is mixed with the at least one CCI.

2. The SCBI according to claim 1, wherein the at least one CCI is arranged on the carrier together with the biological indicator.

3. The SCBI according to claim 1, wherein the at least one CCI is separately arranged inside the vial as at least one pearl or on at least one separate carrier so there is no contact to the biological indicator thereby preventing growth of microorganisms or a negative influence on the long-term stability of the biological indicator on the carrier.

4. The SCBI according to claim 3, wherein the at least one CCI is arranged on an inner surface of a wall of the vial and/or on an outer surface of a wall of the ampule.

5. The SCBI according claim 1, wherein the at least one CCI is inside a second ampule dissolved in a liquid which is placed in the vial together with the ampule of the growth medium.

6. The SCBI according to claim 1, wherein the at least one CCI changes its color due to an enzymatic reaction together with a chemical substance.

7. The SCBI according to claim 1, wherein the ampoule ampule is made from glass and/or the vial is made from a deformable plastic.

8. The SCBI according to claim 1, wherein the carrier is a carrier disc or a strip and/or the carrier is made from paper, stainless steel or glass fiber or any plastic material.

9. The SCBI according to claim 1, wherein the at least one CCI is not arranged inside said ampule or in any other ampule inside said vial.

10. The SCBI according to claim 1, wherein the at least one CCI is a pH-indicator.

11. The SCBI according to claim 1, wherein the at least one CCI is arranged at more than one location inside said vial but outside said ampule.

12. The SCBI according to claim 1, wherein the carrier is arranged inside said vial and outside said ampule.

13. The SCBI according to claim 1, wherein the SCBI is usable for testing sterility achieved by radiation or by gas sterilization.

14. The SCBI according to claim 1, wherein the second color of the at least one CCI changes to an indication color in a reaction to contact with at least one metabolic product from the biological indicator.

15. A method for testing sterility using an SCBI according to claim 1, the method comprising the chronological steps of
A) Activating an ampule transferring a growth medium inside a vial;
B) Bringing the growth medium in contact with a biological indicator and with at least one CCI, wherein a color of the growth medium is changed by the at least one color change indicator;
C) Allowing microorganisms from the biological indicator to grow in the growth medium; and
D) Observing by visual or photometric inspection if a change of color occurs which could be a result of a reaction of the at least one CCI with at least one metabolic product from the microorganism.

16. The method according to claim 15, wherein before method step C) is carried out, controlling if a change of the color of the growth medium occurred and only if the change of color is observed continuing with step C) and otherwise further opening the ampule or deforming the vial to allow mixing of the growth medium with the at least one CCI.

17. The SCBI according to claim 4, wherein the at least one CCI is arranged on an inner surface of a wall of the vial and/or on an outer surface of a wall of the ampule without being in contact with the biological indicator on the carrier.

18. The SCBI according to claim 6, wherein the at least one CCI changes its color due to an enzymatic reaction together with a chemical substance, wherein the color change, an optical density change, or a biofluorescence reaction with UV-light of the at least one CCI is detectable.

19. The SCBI according to claim 7, wherein the ampule is made from a translucent deformable plastic.

20. The SCBI according claim 9, wherein the at least one CCI is arranged without any container inside said vial.

21. The SCSI according to claim 13, wherein the SCSI is usable for testing sterility achieved by UV radiation and gamma radiation, or by ethylene oxide sterilization, formaldehyde sterilization (LTSF), and steam.
